(19) United States
(12) Patent Application Publication
(10) Pub. No.: US 2010/0274120 A1
(43) Pub. Date: Oct. 28, 2010

(54) SYNCHRONOUS INTERVENTIONAL SCANNER

(75) Inventor: Dominic J. Heuscher, Aurora, OH (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., EINDHOVEN (NL)

(21) Appl. No.: 12/746,805

(22) PCT Filed: Dec. 12, 2008
(86) PCT No.: PCT/IB08/55275

§ 371 Date: Jun. 8, 2010

Related U.S. Application Data
(60) Provisional application No. 61/015,822, filed on Dec. 21, 2007.

(51) Int. Cl. A61B 6/00; A61B 6/03

(52) U.S. Cl. 600/424; 378/4; 378/197; 378/150; 378/116

(57) ABSTRACT

When performing an interventional CT scan on a subject, radiation dose is limited by employing a dynamic collimator (142) that collimates X-rays emitted by an X-ray source (112). The X-ray source (112) and collimator (142) rotate around a VOI (122) in the subject, and move axially along the VOI (122) to maintain the tip of a medical instrument (144) within the field of view of the narrow cone beam. An instrument tracking component (146) maintains information related to previous and current positions of the instrument (144) relative to the VOI (122) and facilitates tracking the instrument as it moves through the VOI (122). A user interface (136) superimposes images of a sub-region of the VOI (122) in which the instrument tip is located onto a pre-generated diagnostic image for viewing by an operator, to track the medical instrument (144).

The present application relates generally to imaging systems, particularly involving computed tomography (CT). However, it will be appreciated that the described technique may also find application in other imaging systems, other medical imaging scenarios, or other image data acquisition techniques.

Conventional cone beam CT systems have included multi-slice detectors, which enable such systems to scan larger regions/volumes of interest in shorter periods of time relative to their single-slice system predecessors. Such scanning can be leveraged to quickly scan whole or large portions of organs and improve temporal resolution, such as by a helical scan or a saddle scan.

Several disadvantages exist in conventional CT systems where an operator has to move a patient table during an interventional procedure. For instance, the operator who is performing the procedure needs to move the table manually, either continuously or in increments, which can be uncomfortable and distracting for both the patient and operator. Moreover, safety can be an issue when other equipment is being used next to the moving couch. Additionally, if the couch is being moved, an operator has to be present in the room to perform the couch movement.

The present application provides new and improved CT scanning systems and methods, which overcome the above-referenced problems and others.

In accordance with one aspect, a system for tracking a medical instrument during an interventional procedure using computed tomography (CT) includes an X-ray source on a rotating gantry that moves axially parallel to a volume of interest (VOI) on a stationary subject support, a dynamic collimator positioned between the X-ray source and the VOI and moveable with the X-ray source; and an X-ray detector positioned opposite the X-ray source and collimator to receive X-rays that have passed through the VOI. The X-ray source emits a full beam of X-rays and the dynamic collimator passes a wide cone beam to generate a diagnostic image of the VOI and restricts passage of X-rays to a narrow cone beam having a reduced width to generate images of a sub-region during the interventional procedure.

In accordance with another aspect, a method of tracking a medical instrument during an interventional CT scan includes emitting X-rays from an X-ray source mounted to a gantry and movable axially and rotationally. opening shutter blades on the collimator to permit a portion of an X-ray cone beam to pass through the VOI, and moving the X-ray source and collimator axially parallel to the VOI to maintain a tip of the medical instrument within a field of view of the X-ray cone beam to track the medical instrument.

In accordance with another aspect, a system for minimizing radiation dose while tracking a medical instrument during an interventional CT scan includes means for supporting a subject in a stationary position, means for emitting X-rays to irradiate a portion of a VOI in the subject; means for collimating emitted X-rays into a narrow cone beam, and means for axially and rotationally moving the means for emitting X-rays and the means for collimating along and around the VOI during the interventional CT scan. The system further includes means for detecting X-rays that have passed through the VOI, means for maintaining a tip of the medical instrument in a field of view of the narrow cone beam as the medical instrument traverses the VOI, and means for superimposing reconstructed images of a sub-region of the VOI, in which the tip of the medical instrument is located, onto a pre-generated diagnostic image of the VOI for presentation to a physician. Furthermore, the system includes means for monitoring radiation dose received by the VOI, and means for at least one of providing a warning signal, narrowing the cone beam, shutting of the means for emitting X-rays, and switching to an intermittent fluoroscopic scan mode, when the monitored radiation dose approaches a predefined upper limit.

One advantage is that X-ray dose to a patient is minimized

Another advantage resides in reducing distraction to an operator so that the operator can focus on the interventional procedure itself and not be distracted by having to move the subject support.

Another advantage resides in improved patient comfort.

Another advantage resides in reduced interference from nearby equipment.

Another advantage resides in automatically aligning the CT image with the interventional procedure.

Another advantage resides in reduced power usage during a CT scan.

Still further advantages of the subject innovation will be appreciated by those of ordinary skill in the art upon reading and understand the following detailed description.

The innovation may take form in various components and arrangements of components, and in various steps and arrangements of steps. The drawings are only for purposes of illustrating various aspects and are not to be construed as limiting the invention.

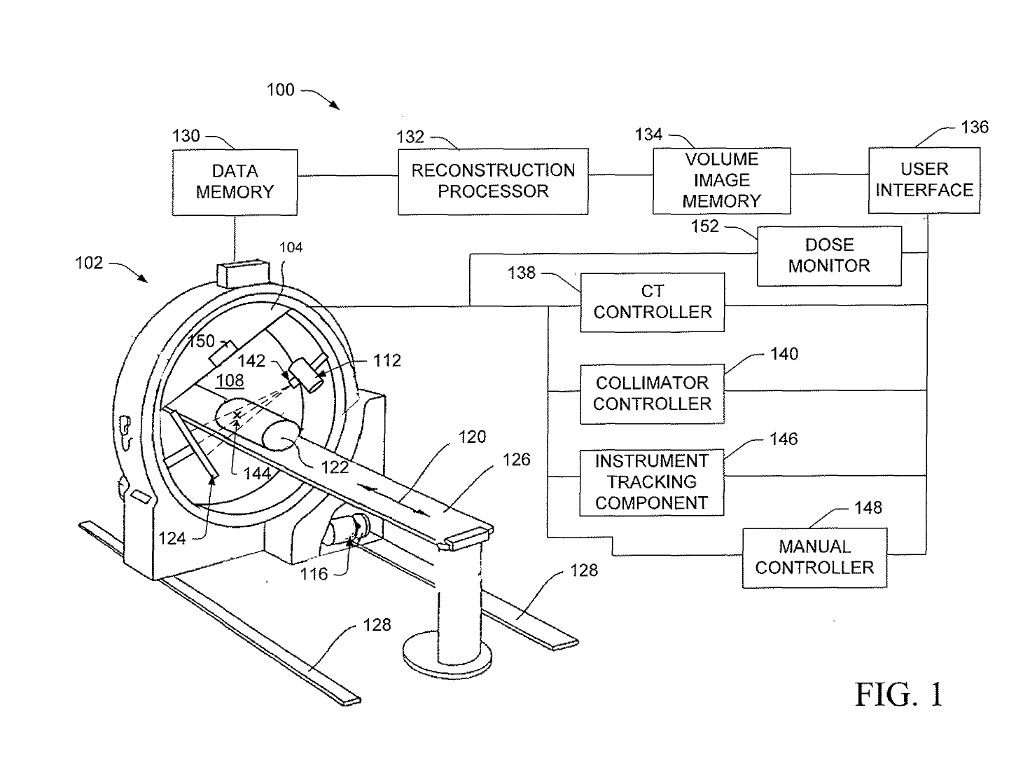

FIG. 1 illustrates a CT imaging system including a CT scanner, such as an interventional scanner or the like, with a rotating gantry portion that rotates about an examination region and moves axially there along.

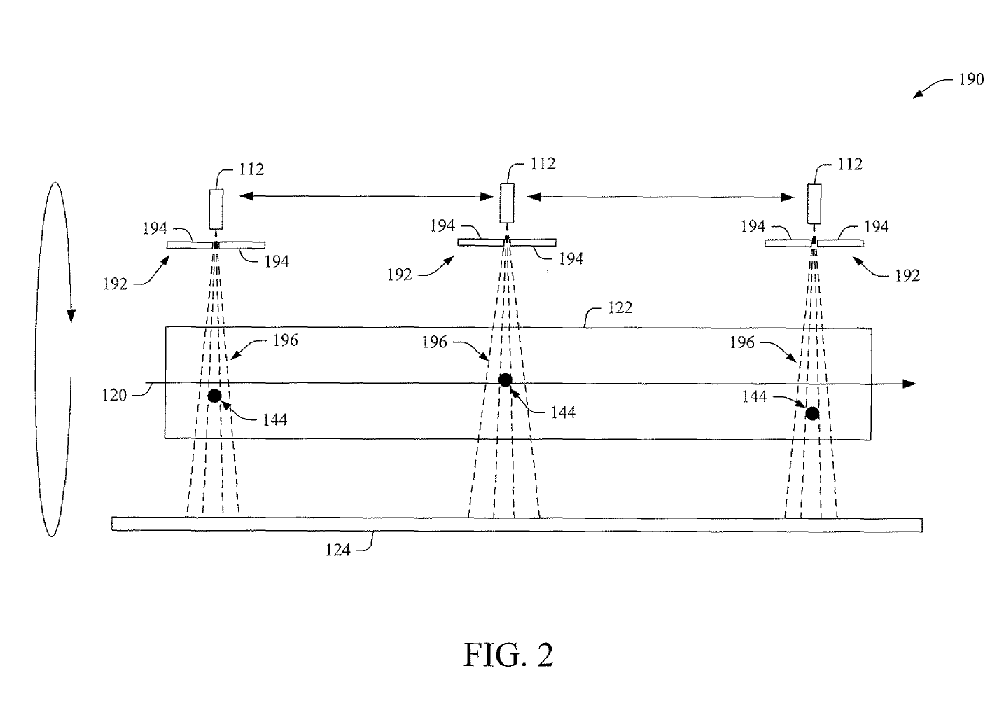

FIG. 2 illustrates a system including a dynamic collimator synchronized to the axial motion of the X-ray source, such as occurs in a saddle scan in which a subject support (FIG. 1) is stationary and the source (or the source/detector combination) moves axially parallel to a z-axis to cover the VOI.

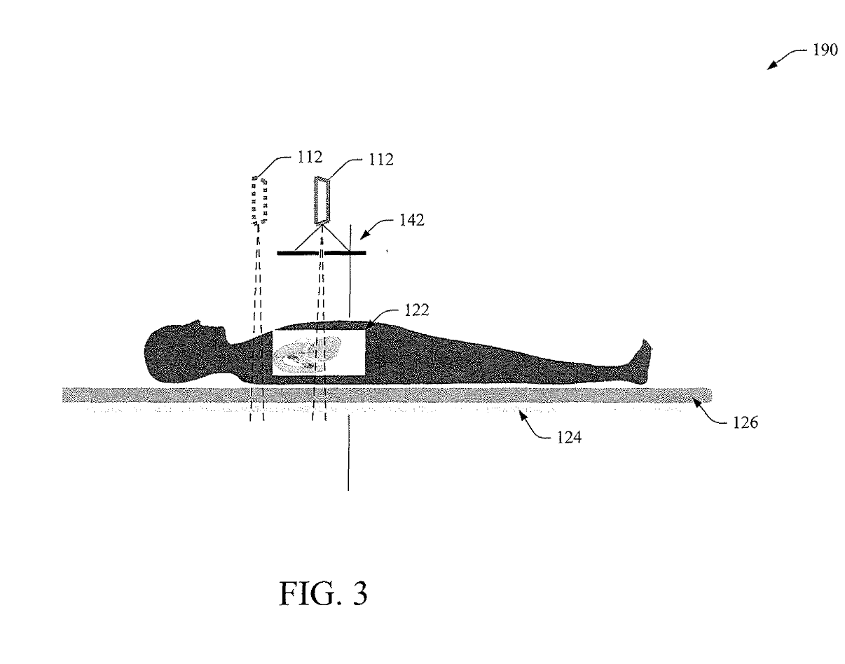

FIG. 3 shows another embodiment of dynamic collimation for interventional CT scans using the system, wherein the dynamic collimator moves with the X-ray source and uses a reduced aperture size for letting X-rays generated by the source pass through the VOI in a narrow (e.g., a few slices) cone or wedge beam to minimize radiation dose to the subject.

According to various aspects, systems and methods described herein relate to tracking the position of a catheter or other medical instrument using computed tomography (CT) to mitigate a need for a doctor or operator to manually move a patient couch in order to retain the tip of the catheter in the CT imaging region. In one embodiment, the CT scanner in which the patient couch remains stationary while an x-ray source on a gantry moves axially and rotationally. An X-ray cone beam is collimated down to a width of a few slices (e.g. 1-2 mm or so) and moved as necessary to track the medical instrument while minimizing radiation dose to the patient.

With reference to FIG. 1, a CT imaging system 100 includes a CT scanner 102, such as an interventional scanner or the like, with a rotating gantry portion 104 that rotates about an examination region 108 and moves axially there along. In one embodiment, the system has a multi-slice detector system 124, for instance, a 128 or 256-slice multi-detector system. Interventional procedures may include procedures performed by a robotic arm or the like. The rotating gantry portion 104 supports an X-ray source 112 (e.g., an X-ray tube) that radiates a cone or wedge X-ray beam that is collimated to have a generally conical or wedge-shaped geometry. A drive mechanism 116 moves the X-ray source longitudinally along a z-axis 120. In one implementation, the motion of the X-ray source and emission of radiation thereby are coordinated to scan a volume of interest (VOI) 122 such as anatomy disposed within the examination region 108 which is optionally enhanced with a contrast agent. As described below, such coordination can be used for, for example, for scanning the VOI during a desired motion state or for tracing the flow of contrast agent through the VOI. Emitted X-rays are then detected at an X-ray detector 124 positioned across the examination region from the X-ray source 112.

The scanner 102 includes a stationary patient support 126 that does not move during the CT acquisition phase while the source or source/detector moves synchronously with the placement of the medical instrument (e.g., a biopsy needle, catheter, or other instrument) used in the interventional procedure. To limit the radiation dose to the patient, the beam is coned down to a width of a few slices (e.g., approximately three or more), which are sufficient for the tracking operation. As an example, the tip of the needle or instrument can be used to control the movement of the X-ray source. A manual override (e.g., a lever or knob) can also be provided for an operator in the event that instrument tracking requires manual operation. Manual tracking can be performed locally or remotely from the scanner.

The rotating gantry portion 104 supports the X-ray sensitive detector array 124, which is disposed about the rotating gantry portion 104 opposite the X-ray source 112. The detector array 124 includes a multi-slice detector having a plurality of detector elements extending in the axial and transverse directions. Each detector element detects radiation emitted by the X-ray source 112 that traverses the examination region 108 and generates corresponding output signals or projection data indicative of the detected radiation. Rather than being arranged in a third generation configuration, other configurations, such as fourth generation configurations in which stationary detectors surround the examination region, are also contemplated herein.

The couch or patient support 126 supports a subject, such as a human patient in which the VOI is defined within the examination region 108. The support 126 is stationary while the rotating gantry 104 is axially movable along tracks 128 that run parallel to the axis 120, which enables an operator of the system to suitably define the VOI to encompass the whole subject or a portion thereof for scanning. In one embodiment, the CT scanner performs a scan of the VOI by rotating around the axis 120 as the X-ray source is moved axially parallel to the z-axis to generate a diagnostic image of the VOI.

The projection data generated by the detector array 124 is stored to a data memory 130 and processed by a reconstruction processor or means 132, which reconstructs the projections and generates a volumetric image representation therefrom. The reconstructed image representation (e.g., a diagnostic image or the like) is stored in a volume image memory 134 and displayed to a user via a user interface 136. The image data is processed to generate one or more images of the scanned region of interest or a subset thereof.

The user interface 136 facilitates user interaction with the scanner 102. Software applications executed by the user interface 136 allow the user to configure and/or control operation of the scanner 102. For instance, the user can interact with the user interface 136 to select scan protocols, and initiate, pause and terminate scanning. The user interface 136 also allows the user to view images, manipulate the data, measure various characteristics of the data (e.g., CT number, noise, etc.), etc.

An optional physiological monitor (not shown) monitors cardiac, respiratory, or other motion of the VOI. In one example, the monitor includes an electrocardiogram (ECG) or other device that monitors the electrical activity of the heart. This information can be used to synchronize saddle scanning with the heart electrical activity. An optional injector (not shown) or the like is used to introduce agents such as contrast into the subject.

The system 100 further includes a CT controller 138, which controls rotational and axial movement of the X-ray source 112 and the X-ray detector 124. The CT scanner and CT controller are additionally coupled to a collimator controller 140 that controls movement, and opening and closing, of a collimator 142 positioned between the X-ray source and the examination region 108. The tip of a medical instrument 144, such as a catheter, a biopsy needle, etc., is maintained in a field of view of the cone beam and tracked by an instrument tracking component 146. The tracking component can include a processor or processors that execute machine-executable algorithms for tracking the instrument tip as it moves through the VOI, and can cooperate with the CT controller 138 and the collimator controller 140 to monitor movement of the instrument tip and move the X-ray source 112 and detector 124 to maintain the narrow cone beam in position to scan the location instrument tip. In the event that the tracking component does not register the instrument tip 144, then the collimator component can widen the collimator aperture to increase the scan area until the instrument tip is located, at which time the cone beam can be narrowed to reduce radiation does to the patient, and tracking is continued.

The detected radiation data from the narrow beam is reconstructed into an image of the subject in a sub-region including the tip of the instrument. The CT controller 138 can cause the X-ray source and detector to conduct an axial scan, by moving the narrow beam across the sub-region in which the tip is located, and optionally the sub-region that the tip is approaching. The scanning distributes the radiation, reducing coverage dose. The image of the sub-region in which the tip is located is reconstructed by the reconstruction processor and displayed on the user interface 136. Multiple images of the sub-region in which the tip is located can be progressively superimposed on the diagnostic image of the VOI to permit a physician or operator to track the tip of the medical instrument.

In one embodiment, sub-region images are superimposed on a pre-generated diagnostic image of the VOI to permit a physician to track movement of the tip. Alternatively, a wider cone beam can be utilized to image the entire sub-region every half-revolution or so. In another embodiment, motion correction can be performed during the axial scan by moving the source and interpolating data to correct for skew, as is performed in a helical scan.

To reduce radiation dose, the X-ray source can be operated in fluoroscopic mode. When the X-ray source and detector rotate at a speed of, for instance, 240 rpm, 6 images are generated per second. If the tip is moving slowly through the subject, the X-ray beam may be gated on/off to generate images more slowly (e.g., 1 image per second). The sub-region image can be combined with or superimposed on the high-resolution image from the image memory 134. In one embodiment, only the tip of the medical instrument is superimposed. In another embodiment, the path of the instrument tip is superimposed, and updated as newly generated images of the tip sub-region become available.

In one embodiment, the X-ray source 112 (or X-ray source 112/collimator 142/detector 124 combination) are moved using an external control, either automatically in coordination with a detected position of the of the medical instrument tip 144 or manually using a manual controller 148 such as a control lever or knob with motion limits corresponding to the axial movement limits of the tube or tube/collimator/detector motion. To limit dose to the patient, the beam is coned down to 3 or more slices, which is sufficiently wide to allow tracking of the interventional instrument. This increases the range over which the source or source/detector can move, which may cover up to, for example, 30 cm or more (e.g., sufficient for interventional procedures) for the volume image and 3 cm for the sub-region image of the tip. The control device can be located either or both inside the scan room or in the control room. Automatic operation can be overridden at any moment by an operator. If an interventional robotic arm is being used, the controls for the arm and the movement of the source or source/detector can be combined. No movement of the couch is thus necessary during the entire procedure.

Additionally or alternatively, a dose monitor 152 monitors total radiation dose received by the VOI 122, and can trigger the collimator 142 to reduce X-ray cone beam width to reduce, change to an intermittent fluorescent mode (e.g., wherein the cone beam is gated on/off with a reduced duty cycle to reduce radiation dose), decrease gating duty cycle, eliminate radiation dose, or the like, as the total radiation dose approaches a predefined upper limit. Furthermore, the dose monitor 152 can provide a warning signal to the operator that the total radiation dose is approaching the upper limit. In one embodiment the dose monitor evaluates reconstructed image data to monitor dose received by each reconstructed pixel.

In another embodiment, the collimator controller causes the collimator to open to a small predefined diameter, which exposes the VOI to an X-ray cone beam that is much smaller than would be used to scan the entire VOI, as the rotatable gantry 104 (and accordingly the source 112 and detector 124 coupled thereto) moves axially along the VOI 122. The collimator controller 140 may include an electro-mechanical servo motor and/or an electronic controller or the like. By limiting the collimator aperture to the small predefined diameter, the VOI receives less than a full cone beam of X-rays, thereby reducing the X-ray dose received by the VOI.

In another embodiment, the CT scanner 102 can include suitable sensors 150 (e.g., infrared sensors, camera sensors, etc.) to detect VOI position information that can be used by the instrument tracking component, the collimator controller, and/or the CT controller to coordinate X-ray source positioning, X-ray beam collimation, and/or instrument tracking.

In another embodiment, the tip of the medical instrument 144 is tracked with a marker that is identifiable in the CT reconstruction, by recognizing the tip of the instrument in the CT reconstruction, with an RF marker, or the like. Based on the determined location of the tip of the medical instrument relative to the current CT imaging field, the CT scanner gantry is shifted to maintain the instrument centered (or at another fixed location) in the imaging field of view.

Other embodiments include minimizing radiation dose to a patient by operating the X-ray source 112 in a fluoroscopic mode, and/or at intervals. For example, the X-ray source can rotate at a high speed (e.g., approximately 220 rpm), and because catheters are moved relatively slowly during insertion, an image of a current location of the tip may only need to be generated approximately once per second. For instance, the X-ray source is flashed on and off as it rotates at high speed (e.g., 220 rpm or greater) around the VOI to further reduce the radiation dose received by the VOI.

In another variation, a detailed 3D volume image of the region into which the medical instrument will be inserted is generated in advance. During the medical instrument insertion, a relatively low dose examination can be performed to monitor the position of the tip of the medical instrument. When the position of the medical instrument is superimposed on the prior reconstructed image, the tip of the catheter can be monitored with a relatively low dose X-ray beam which produces relatively noisy images. In a variation, a relatively high resolution image of the region of the patient downstream from the medical instrument tip is built from multiple revolutions of the prior medical instrument tracking data.

In another embodiment, the radiation dose per voxel is monitored. A dosage readout can be provided, a warning can be provided when the dose reaches or exceeds a predetermined level, the fan or cone beam can be narrowed, a duty cycle of the x-ray beam can be reduced, or the like.

In yet another embodiment, the medical instrument is inserted over a longer axial distance than the range of axial movement of the x-ray source 112, and the process is performed in phases with the patient support moved between phases.

FIG. 2 illustrates a system 190 including a dynamic collimator 142 synchronized to the axial motion of the X-ray source 112, such as occurs in an axial scan in which a subject support (FIG. 1) is stationary and the source 112 (or the source 112/detector 124 combination) moves axially parallel to a z-axis 120 to cover the VOI 122. The collimator includes at least two high-speed shutter or collimator blades 194 that are independently adjustable to define a collimator aperture through which X-rays are permitted to pass to generate a cone or fan beam 196. The collimator can be fixed to a rotate plate on the CT scanner gantry or attached to the source 112 itself. The collimator 142 and source 112 are illustrated in several positions along the volume of interest. Although described and depicted in a linear trajectory, it will be understood that the collimator and source may rotate around the VOI as they move axial along the VOI, in a helical trajectory, if desired. In another embodiment, the source can also move back and forth along helical paths to conduct a saddle scan of a selected region or sub-region. In one embodiment, the detector moves around the VOI, opposite the source and collimator. In another embodiment, the detector is cylindrical and surrounds the VOI and source/collimator in order to detect X-rays emitted from the source regardless of the rotational position of the source relative to the VOI. That is, the X-ray detector 124 can be stationary both axially and rotationally, as in a fourth generation system, or can be movable to travel with the X-ray source and collimator along the volume of interest parallel to the z-axis and opposite the X-ray source. In another embodiment, the X-ray detector is stationary relative to the z-axis and rotatable around the VOI opposite the X-ray source. In the case of a moveable detector, an anti-scatter grid can be optionally employed to improve image reconstruction quality and reduce radiation dose to the patient.

In one embodiment, the collimator 142 is attached to the source 112. In another embodiment, such as where the collimator is positioned close to the volume of interest, the collimator 142 may be separate from the source 112. In this manner, the collimator reduces X-ray dose to the patient by limiting the width of the X-ray cone beam to only a few slices (e.g., 3, 4, 5, 8, 12, etc.), which is sufficient to track the medical instrument tip 144.

FIG. 2 thus illustrates exemplary motion of the X-ray source 112 along the z-direction and corresponding X-ray beam geometry. While translating along the VOI 122, the X-ray source 112 rotates around the examination region (FIG. 1) and emits X-rays. The X-ray source 112 may also move bi-directionally parallel to the z-axis, for example, when performing the initial scan, a subsequent scan, or a saddle scan of the sub-region adjacent the tip.

FIG. 3 shows another embodiment of dynamic collimation for interventional CT scans using the system 190, wherein the dynamic collimator 142 moves with the X-ray source 112 and maintains a constant aperture size for letting X-rays generated by the source 112 pass through the VOI 122 in a narrow (e.g., a few slices) cone or wedge beam to minimize radiation dose to the subject. The subject support 126 remains stationary during the interventional scan while the source 112, collimator 142, and detector 124 rotate around the VOI as the source and collimator move axially along the VOI. In one embodiment, the detector is stationary relative to the z-axis and rotates with the source and collimator maintaining a substantially opposite (e.g., 180°) orientation thereto. In another embodiment, the detector is cylindrical and surrounds the subject, VOI, source, and collimator to receive collimated X-rays regardless of the axial or rotational orientation of the source and collimator, such as in a fourth-generation CT system. According to an example, the X-ray source 112 is a 128-slice or 256-slice source, and the collimator 142 permits a cone or wedge beam of only a few slices (e.g., 3, 4, 8, etc.) to pass through to the VOI 122.

The innovation has been described with reference to several embodiments. Modifications and alterations may occur to others upon reading and understanding the preceding detailed description. It is intended that the innovation be construed as including all such modifications and alterations insofar as they come within the scope of the appended claims or the equivalents thereof.

1. A system for tracking a medical instrument during an interventional procedure using computed tomography (CT), including:
an X-ray source on a rotating gantry that moves axially parallel to a volume of interest (VOI) on a stationary subject support;
a dynamic collimator positioned between the X-ray source and the VOI and moveable with the X-ray source; and
an X-ray detector positioned opposite the X-ray source and collimator to receive X-rays that have passed through the VOI;
wherein the X-ray source emits a full beam of X-rays and the dynamic collimator passes a wide cone beam to generate a diagnostic image of the VOI and restricts passage of X-rays to a narrow cone beam having a reduced width to generate images of a sub-region during the interventional procedure.

2. The system according to claim 1, wherein the collimator is coupled to one of:
a rotator plate on a rotatable gantry to which the X-ray source is attached; and
the X-ray source.

3. The system according to claim 1, further comprising a manual controller that permits manual control of movement of the X-ray source and collimator along the VOI to track the medical instrument during the interventional procedure.

4. The system according to claim 3, wherein the manual controller is at least one of a knob or a lever, with motion limits corresponding to the limits of axial motion of the X-ray source.

5. The system according to claim 1, wherein the gantry is configured to move back and forth along the z-axis to scan the VOI to perform at least one of a saddle scan and a repeated fly-by scan.

6. The system according to claim 1, further comprising:
an instrument tracking component that processes CT scan data to determine a position of the medical instrument being tracked by the system.

7. The system according to claim 6, further comprising:
a sensor that senses a position of the VOI; and
a collimator controller that controls an aperture size of the collimator as a function of the relative positions of the VOI, the medical instrument, and the X-ray source.

8. The system according to claim 7, further including a radiation dose monitor that monitors total radiation dose received by the VOI and triggers the collimator controller to at least one of shut off x-rays, provide a warning signal, switch to an intermittent fluoroscopic scan mode, and narrow the cone beam, as the total radiation dose approaches a predefined upper limit.

9. The system according to claim 6 wherein the tracking component generates an image of a sub-region of the VOI in which the tip of the medical instrument is located.

10. The system according to claim 9, wherein the user interface superimposes the image of the tip of the medical instrument on a previously generated diagnostic image region of the VOI.

11. A method of tracking a medical instrument during an interventional CT scan using the system of claim 1, including:
emitting X-rays from the X-ray source mounted to the gantry and movable axially and rotationally;
opening shutter blades on the collimator to permit a portion of an X-ray cone beam to pass through the VOI; and
moving the X-ray source and collimator axially parallel to the VOI to maintain a tip of the medical instrument within a field of view of the X-ray cone beam to track the medical instrument.

12. A method of tracking a medical instrument during an interventional CT scan, including:
emitting X-rays from an X-ray source mounted to a gantry and movable axially and rotationally;
opening shutter blades on the collimator to permit a portion of an X-ray cone beam to pass through the VOI; and
moving the X-ray source and collimator axially parallel to the VOI to maintain a tip of the medical instrument within a field of view of the X-ray cone beam to track the medical instrument.

13. The method according to claim 12, further including dynamically adjusting the shutter blades to widen or narrow the cone-beam while tracking the tip of the medical instrument.

14. The method according to claim 13, further including:
reconstructing acquired CT scan data to verify a position of the medical instrument in the VOI and to determine whether to adjust the width of the cone beam.

15. The method according to claim 14, further comprising reconstructing and displaying a sub-region of the VOI including the tip of the medical instrument.

16. The method according to claim 15, further including manually moving the gantry axially along the VOI during the interventional CT scan to maintain the tip of the medical instrument within the field of view of the cone beam.

17. The method according to claim 15, further including automatically moving the gantry axially along the VOI, as a function of CT scan data, VOI position, or medical instrument position, during the interventional CT scan to maintain the tip of the medical instrument within the field of view of the cone beam.

18. The method according to claim 14, further including monitoring radiation dose to the VOI and at least one of sending a warning signal, shutting off X-rays, narrowing the width of the cone beam, and switching to an intermittent fluoroscopic scan mode, when the radiation dose approaches a predefined upper limit.

19. The method according to claim 12, further including generating a diagnostic image of the VOI with a wide X-ray cone beam.

20. The method according to claim 19, further including:
reconstructing a sub-region of the VOI with a narrow X-ray cone beam to generate a series of sub-region images; and
combining the sub-region images with the diagnostic image to show a current position of the tip of the medical instrument relative to the diagnostic image.

21. The method according to claim 20, further including moving the narrow cone beam back and forth to image the sub-region while performing at least one of a saddle scan and a repetitive fly-by scan.

22. The method according to claim 20, moving the X-ray source in an intermittent fluoroscopic mode, wherein a contrast agent is introduced into the subject.

23. The method according to claim 20, further including:
monitoring radiation dose to the subject; and
in response to the monitored radiation dose approaching a pre-selected radiation limit, at least one of:
switching the X-ray source to an intermittent fluoroscopic mode;
narrowing the cone beam;
emitting a warning signal; and
shutting off the X-ray source.

24. A system for minimizing radiation dose while tracking a medical instrument during an interventional CT scan, including:
means for supporting a subject in a stationary position;
means for emitting X-rays to irradiate a portion of a VOI in the subject;
means for collimating emitted X-rays into a narrow cone beam; means for axially and rotationally moving the means for emitting X-rays and the means for collimating along and around the VOI during the interventional CT scan;
means for detecting X-rays that have passed through the VOI;
means for maintaining a tip of the medical instrument in a field of view of the narrow cone beam as the medical instrument traverses the VOI;
means for superimposing reconstructed images of a sub-region of the VOI, in which the tip of the medical instrument is located, onto a pre-generated diagnostic image of the VOI for presentation to a physician;
means for monitoring radiation dose received by the VOI; and
means for at least one of providing a warning signal, narrowing the cone beam, shutting of the means for emitting X-rays, and switching to an intermittent fluoroscopic scan mode, when the monitored radiation dose approaches a predefined upper limit.
